(19) United States
(12) Patent Application Publication
(10) Pub. No.: US 2025/0241746 A1
(43) Pub. Date: Jul. 31, 2025

(54) CAPSULAR TENSION RING INSERTION DEVICE AND METHOD OF USE

(71) Applicant: Corza Ophthalmology Inc, Parsippany, NJ (US)

(72) Inventors: Takahiro Shimowake, Matsuyama (JP); Takeuchi Mamoru, Hiroshima (JP); Abhishek Gundugurti, Chicago, IL (US); Sagar Ghotavadekar, Elk Grove Village, IL (US)

(21) Appl. No.: 19/176,875

(22) Filed: Apr. 11, 2025

Related U.S. Application Data
(63) Continuation of application No. PCT/US2023/076685, filed on Oct. 12, 2023.
(60) Provisional application No. 63/415,780, filed on Oct. 13, 2022.

(51) Int. Cl. A61F 2/16; A61F 9/00

(52) U.S. Cl. CPC A61F 2/1694; A61F 9/0017

(57) ABSTRACT

An insertion device for inserting a capsular tension ring into the intraocular space of a patient's eye during cataract surgery includes a main body and a tube extending from a first end of the main body. The device also has an external hook attached to an outside surface of the tube at a first end of the tube which is farthest away from the main body, and an internal hook attached to a push mechanism. The external hook extends beyond the first end of the tube and the internal hook is moveable with respect to the tube. When the push mechanism is in a first position, the internal hook is within the tube, and when the push mechanism is in a second position, the internal hook extends beyond the first end of the tube.

### CROSS REFERENCE TO RELATED APPLICATIONS

This application is a continuation of International Application No. PCT/US2023/076685, filed on Oct. 12, 2023, which claims priority to U.S. Provisional Patent Application No. 63/415,780, filed on Oct. 13, 2022, the contents of which are hereby incorporated in their entirety.

### TECHNICAL FIELD

The present invention relates generally to a capsular tension ring insertion device and a corresponding method of inserting a capsular tension ring into a subject's eye.

### BACKGROUND

A capsular tension ring can be very useful in stabilizing a subject's lens capsule (or capsular bag) during eye surgery, such as cataract removal. Capsular tension rings are often made from polypropylene or other similar material, and are usually made of clear material, thus having no coloration. Typical capsular tension rings have a ring-like shape and a diameter that allows a hook of an inserter device to fit inside the ring in a snug manner.

Patients undergoing cataract surgery sometimes require a support mechanism to support the capsular bag in the anterior segment of the eye. A capsular tension ring is commonly used as this support mechanism. In some instances, the fragile Zinn's membrane makes it difficult to insert a capsular tension ring into the intraocular space without placing stress on the capsular bag, which could cause it to tear. Known capsular tension ring insertion devices used during capsulorhexis are difficult to use and typically require the use of both hands to maneuver the device to appropriately place the capsular tension ring in the correct location of the capsular bag. This bag is fragile, and any additional stress is prone to cause issues. Thus, there is a clear and substantial need for a capsular tension ring insertion device that is operable to safely and easily insert a capsular tension ring into the intraocular space within the eye of a patient during cataract surgery.

The capsular tension ring insertion device of the present invention avoids these aforementioned problems. In particular, the capsular tension ring insertion device of the present invention is operable to perform a spiral insertion method for placing the capsular tension ring into the intraocular space. The spiral insertion method advantageously lets an operator insert a capsular tension ring into a capsular bag without stressing the Zinn's membrane, since it allows the capsular tension ring to release from a corresponding insertion device slowly as the capsular tension ring is inserted, thus allowing the capsular tension ring to conform with the shape of the capsular bag as the capsular tension ring expands. As a result, unnecessary stress on the capsular bag is avoided. However, such a method of insertion of a capsular tension ring can be very difficult for many surgeons who perform cataracts surgery. This difficulty is exacerbated by known capsular tension ring inserters, which are difficult to operate-especially with only one hand. The present invention solves these problems by providing a capsular tension ring insertion device that is easy, safe, and effective for inserting a capsular tension ring into the intraocular space in the eye of a patient during cataract surgery. Thus, the capsular tension ring insertion device and method of the present invention is easy to use and can be readily used with one hand, while avoiding stress on the capsular bag during the procedure.

### SUMMARY

The present invention relates to a capsular tension ring insertion device for inserting a capsular tension ring into the intraocular space of a patient's eye during cataract surgery. The capsular tension ring insertion device includes a main body and a tube extending from a first end of the main body. The device also has an external hook attached to an outside surface of the tube at a first end of the tube which is farthest away from the main body and an internal hook attached to a push mechanism. The external hook extends beyond the first end of the tube and the internal hook is moveable with respect to the tube. Finally, when the push mechanism is in a first position, the internal hook is within the tube and when the push mechanism is in a second position, the internal hook extends beyond the first end of the tube.

According to one aspect, the capsular tension ring insertion device has the internal hook extend beyond the first end of the tube more than the external hook. Further, a spring is positioned within a cavity of the main body as part of the push mechanism. It is configured in such a way to be in an uncompressed state when the push mechanism is in the first position and a compressed state when the push mechanism is in the second position.

According to another aspect, the capsular tension ring insertion device has the internal and external hooks configured to be inserted into an end of the capsular tension ring, in particular into an eyelet to guide and hold onto the capsular tension ring during insertion and allow for easy removal. The shapes of the internal and external hooks can vary and may be any shape capable of insertion into an eyelet of a capsular tension ring known in the art including a fish-hook, C-, or L-shape. Additionally, to ensure stability of the capsular tension ring during insertion, the tips (i.e., the first and second eyelet grips) of the internal and external hooks may include a ball shape, tear-drop shape, or a loop.

According to one aspect, the tube has a curvature to allow easier access for the capsular tension ring to enter the intraocular space. This curvature has a radius of 5 mm to 30 mm. In another variant, the external hook and the internal hook form an angle of 5° to 45° when the internal hook is extended in the second position. This angle allows for easier maneuverability of the capsular tension ring during insertion.

The present invention also relates to the method of inserting a capsular tension ring into an intraocular space of a patient's eye using the insertion device. The steps of which generally proceed as follows:

First, a user, such as a licensed physician, presses on the push mechanism of the device to extend the internal hook of the device from the first position to the second position beyond the end of a tube of the device. Next, the user inserts the internal hook into a first eyelet of the capsular tension ring. Then, the user pulls back on the push mechanism to withdraw the internal hook and the capsular tension ring into the tube until the internal hook and the first eyelet are at a position approximately halfway up the tube. The user then inserts external hook of the device into a second eyelet of the capsular tension ring. Following, the user pulls on the push mechanism to withdraw the internal hook and the capsular tension ring into the tube until the internal hook is in the first position and places a tip of the external hook into the intraocular space of the eye. Finally, the user presses on the push mechanism to extend the internal hook and the first eyelet to the second position and tilts the device to release the capsular tension ring from the device.

There has thus been outlined certain embodiments of the present invention in order that the detailed description thereof herein may be better understood, and in order that the present contribution to the art may be better appreciated. Details associated with these embodiments as well as additional embodiments of the present invention will be described below, and which form the subject matter of the claims appended hereto.

In this respect, before explaining at least one aspect of the capsular tension ring insertion device in detail, it is to be understood that the capsular tension ring insertion device is not limited in its application to the details of construction and to the arrangements of the components set forth in the following description or illustrated in the drawings. The capsular tension ring insertion device is capable of aspects in addition to those described, and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the capsular tension ring insertion device. It is important, therefore, that the claims be regarded as including such equivalent constructions insofar as they do not depart from the spirit and scope of the invention.

### BRIEF DESCRIPTION OF THE DRAWINGS

In order that the present invention may be readily understood, aspects of the capsular tension ring insertion device of the present invention are illustrated by way of examples in the accompanying drawings, in which like parts are referred to with like reference numerals throughout.

FIG. 1 shows a top view of a capsular tension ring insertion device a first position according to the present disclosure.

FIG. 2 shows a top view of the capsular tension ring insertion device of FIG. 1 in a second position.

FIG. 3 shows a side view of the capsular tension ring insertion device of FIG. 1 in the second position.

FIG. 4 shows a cross-sectional top view of the capsular tension ring insertion device of FIG. 1 in the first position.

FIG. 5 shows a front view of the capsular tension ring insertion device of FIG. 1 in the second position.

FIG. 6A shows a top left perspective view of a distal end of the capsular tension ring insertion device of FIG. 1 in the second position, including external and internal hooks.

FIG. 6B shows a top right perspective view of a distal end of the capsular tension ring insertion device of FIG. 1 in the second position, including external and internal hooks.

FIG. 6C shows a top view of a distal end of the capsular tension ring insertion device of FIG. 1 in the second position, including external and internal hooks.

FIG. 6D shows the external and internal hooks from a cross-sectional side view along the X-X axis of FIG. 6C.

FIG. 7A shows another implementation of the external and internal hooks at the distal end of the capsular tension ring insertion device.

FIG. 7B shows another implementation of the external and internal hooks at the distal end of the capsular tension ring insertion device.

FIG. 7C shows another implementation of the external and internal hooks at the distal end of the capsular tension ring insertion device.

FIG. 8 shows a capsular tension ring for use with the capsular tension ring insertion device of the present disclosure.

FIG. 9 depicts a spiral insertion method for inserting a capsular tension ring into a subject's intraocular space using the capsular tension ring insertion device of the present disclosure.

### DETAILED DESCRIPTION

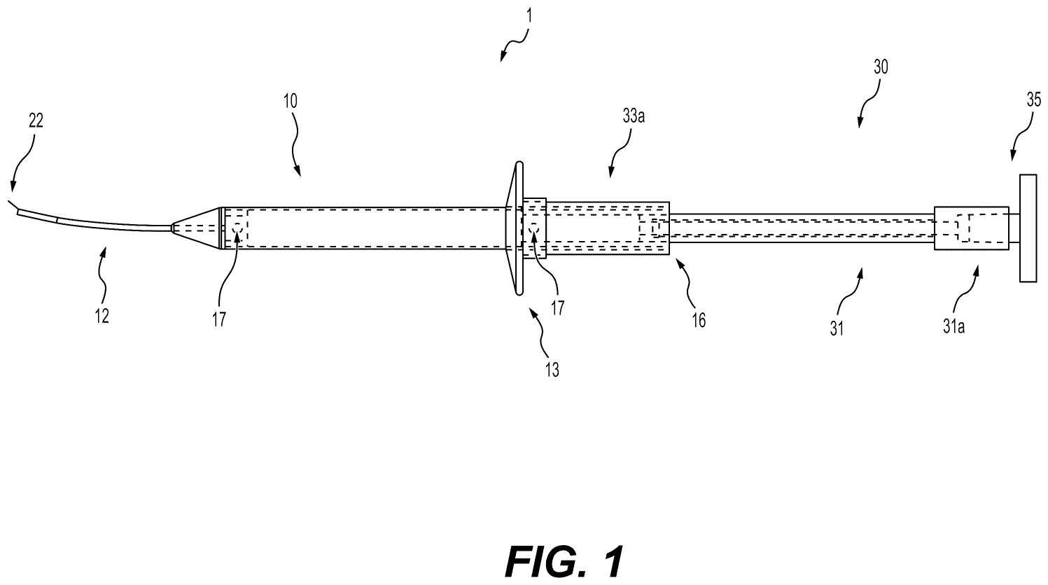

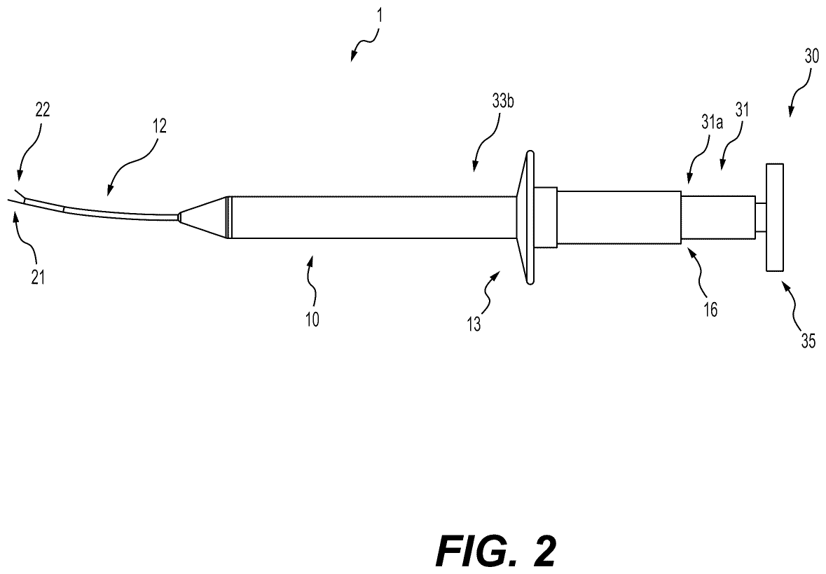

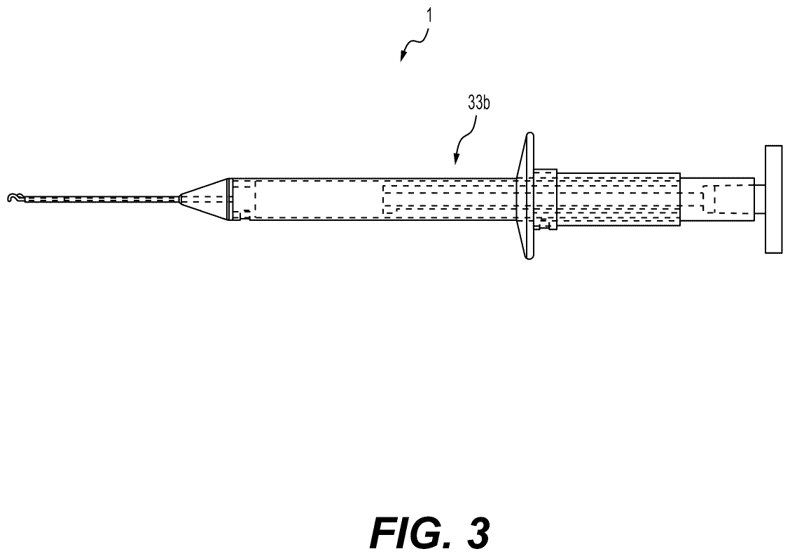

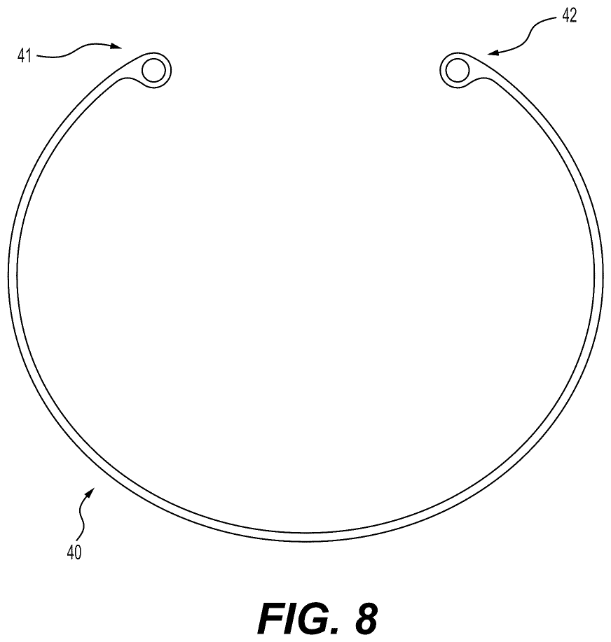

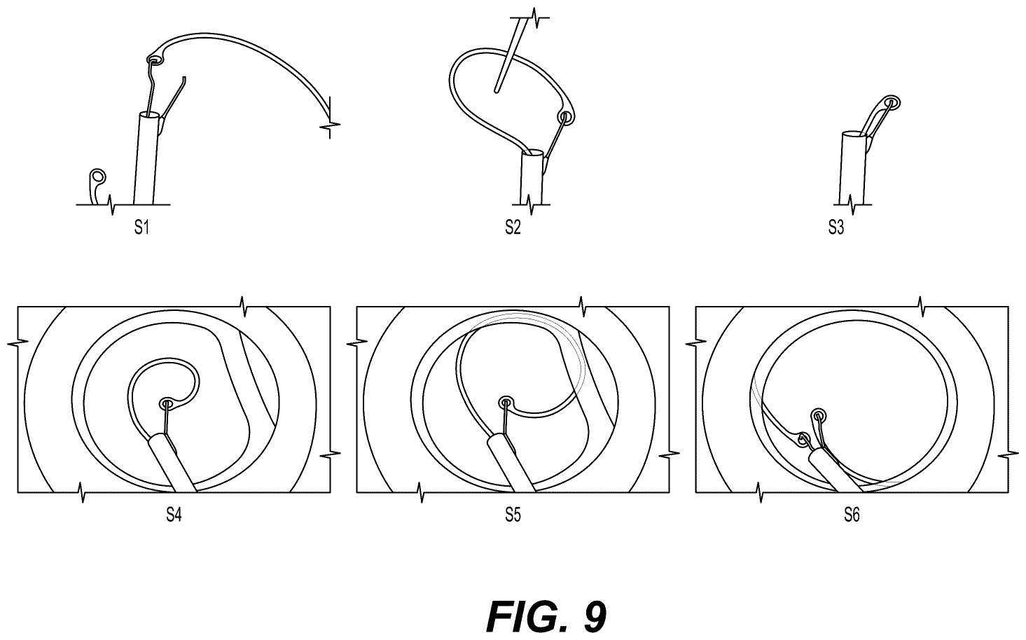

A capsular tension ring insertion device 1, as seen in FIGS. 1-4, includes a main body 10 having a first end and a second end, where a tube 12 extends from the first end of the main body 10. The capsular tension ring insertion device 1 also has an external hook 22 attached to an outside surface of the tube 12 at a first end of the tube 12 which is farthest away from the main body 10 and an internal hook 21 attached to a push mechanism 30. The capsular tension ring insertion device 1 is configured to insert a capsular tension ring 40 which has a pair of spaced apart eyelets (41, 42), as seen in FIG. 8, into the capsular bag of a patient's eye.

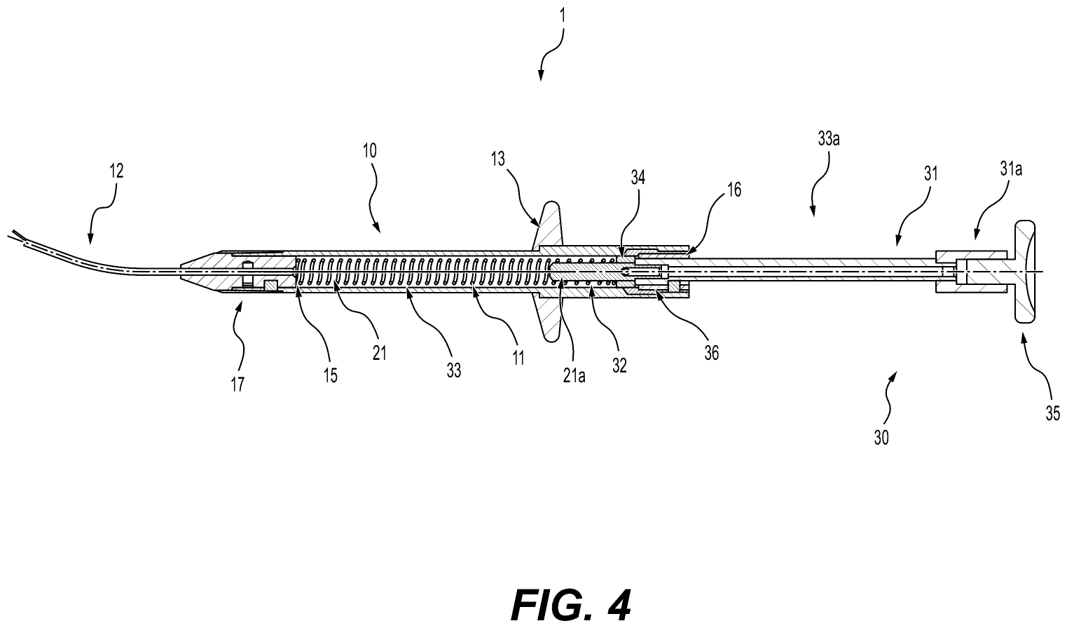

The external hook 22 is fixed or attached at or near the end of the tube 12. On the other hand, the internal hook 21 has a proximal end 21b (seen in FIG. 4) which is connected to a carriage member 32 of the pushing mechanism which slides along the length of the main body 10 with the internal hook 21. This allows the internal hook 21 to move the capsular tension ring in and out of the capsular tension ring insertion device 1 for easy insertion into the intraocular space. The tube 12 itself has a bore 12a configured to receive the capsular tension ring, where the tube's distal end has an opening (i.e., at the end farthest away from the main body 10) that allows for the internal hook 21 to receive an eyelet 42 of the capsular tension ring and withdraw it inside the tube 12 and/or the main body 10. The tube 12 preferably has a gauge of 18G (1.1 mm) to 22G (0.7 mm). The capsular tension ring is flexible so that it can bend to allow it to fit within the tube 12 and/or the main body 10.

In one aspect of the invention, the tube 12 has a curvature to allow easier access for the capsular tension ring to enter the intraocular space during the procedure. For example, this curvature has a radius of 5 mm to 30 mm and is designed to approximately match the curvature of the temporal or nasal regions of the patient. However, the tube 12 may also be straight. The tube 12 preferably has a length between 19 mm and 27 mm. The tube 12 may be assembled to the main body 10 using one or more screws 17. Further, the tube may be made from stainless steel.

The internal hook 21 is also flexible so it can readily move through the main body 10 and the tube 12 with ease to guide the capsular tension ring during the procedure. Some suitable materials for the external and the internal hooks 21 include stainless-steel, aluminum, and biocompatible plastic. Examples of biocompatible plastics include polyoxymethylene (Delrin®), polyethylene, polycarbonate, and polyethylene terephthalate. Other components of the capsular tension ring insertion device 1 may also be made of these exemplary materials. However, any material obvious to a person of ordinary skill in the art suitable for a capsular tension ring insertion device 1 could be used.

One variant of the main body 10 is a hollow cylinder made of metal, but may also have various cross-sections including a square, rectangle α, or other polygonal shapes. The main body 10 may be made of aluminum, stainless steel, titanium, or a biocompatible plastic. Some suitable biocompatible plastics include polyoxymethylene (Delrin®), polyethylene, polycarbonate, and polyethylene terephthalate, among others. However, any material obvious to a person of ordinary skill in the art suitable for a capsular tension ring insertion mechanism could be used. The main body 10 may have a diameter between 4.5 mm and 7 mm.

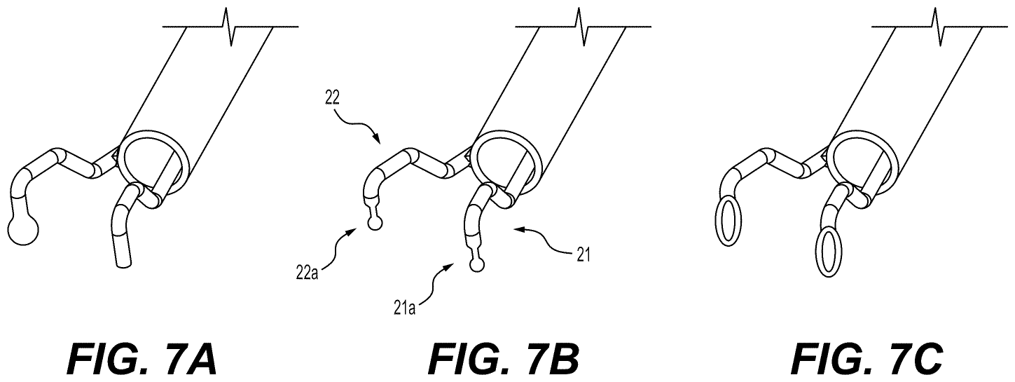

The internal hook 21 and external hook 22 are also configured to be inserted into an end of the capsular tension ring 40 into respective eyelets (41, 42, seen in FIG. 8) to guide and hold onto the capsular tension ring during insertion and allow for easy removal. As a result, a distal end of the external hook defines a first eyelet grip 22a and a distal end of the internal hook defines a second eyelet grip 21a. As seen in FIGS. 7A-7C, the shapes of the internal hook 21 and external hook 22 can vary and may be any shape capable of insertion into an eyelet (41, 42) of a capsular tension ring known in the art including a fish-hook, C-, or L-shape. Additionally, to ensure stability of the capsular tension ring during insertion, the tips (i.e., the first and second eyelet grips 21a, 22a) of the internal hook 21 and external hook 22 may include a ball shape, tear-drop shape, or a loop.

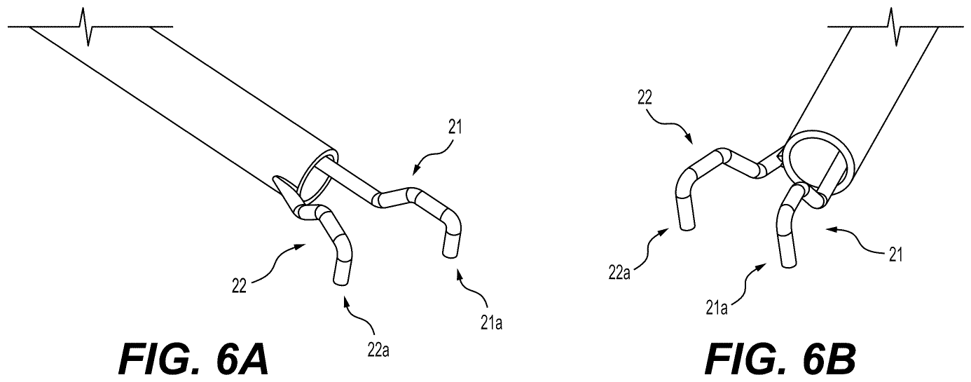

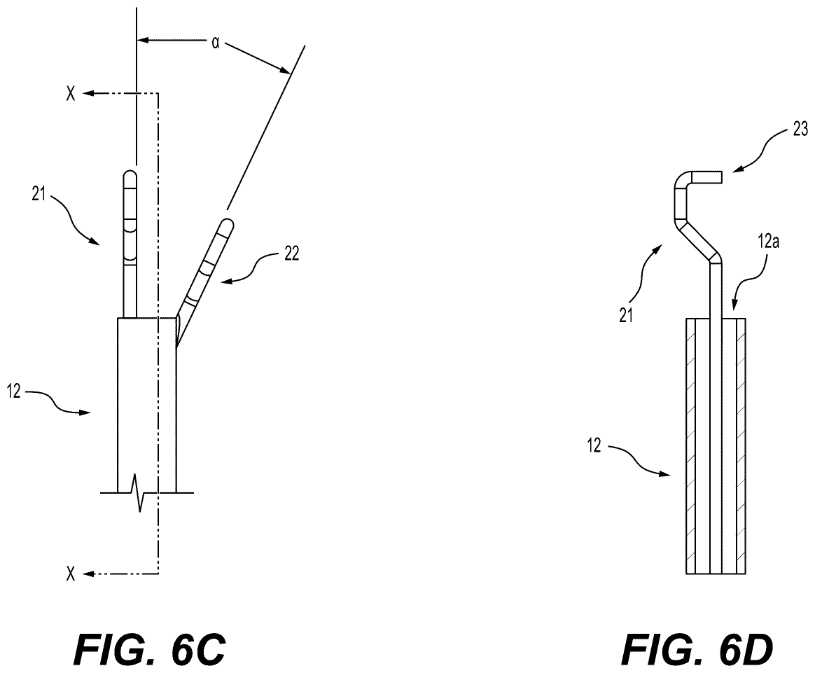

As seen in FIGS. 6A-6D, according to another aspect of the invention, the external hook 22 and the internal hook 21 form an angle α of 5° to 45° between each other when the internal hook 21 is fully extended outside of the tube 12 (e.g., when the push mechanism is in the second position). Preferably this angle α is 15° to 30° and even more preferably this angle α is 20°. This angle α allows for easier maneuverability of the capsular tension ring during insertion when using a spiral insertion method. During insertion of the capsular tension ring, the external hook 22 extends beyond the distal end of the tube 12 by a first distance of 1 mm to 4.5 mm and preferably between 1 mm and 2 mm.

In contrast, the internal hook 21 extends beyond the distal end of the tube 12 by a second distance between 1 mm and 4 mm, but preferably, this distance is 2 mm to 4 mm. Stated another way, the second distance is preferably greater than the first distance. The preferable lengths of the internal hook 21 and the external hook 22 results in the internal hook 21 preferably extending beyond the external hook 22 when the push mechanism 30 in its fully pressed position because this creates the optimal holding position for the physician to place the capsular tension ring appropriately in the patient's intraocular space during cataract surgery. That is, preferably, the second distance is greater than the first distance.

In another aspect of the invention, seen in FIG. 4, the capsular tension ring insertion device 1 has a push mechanism 30, which allows the user to easily insert the capsular tension ring into a patient's intraocular space with one hand. The push mechanism 30 includes an actuator including a plunger 31, the carriage member 32, a shoulder member 34, which also operates as a washer, a linear bushing 36, and a removable thumb rest 35 fitted on the tip of the plunger 31a. Additionally, the carriage member 32 is movably disposed within the main body and connected to the actuator of the push mechanism 30. Further, the plunger 31 has a distal end connected to the carriage member 32, and a proximal end connected to a push member. The push mechanism 30 is also in cooperation with a spring 33 positioned within a cavity 11 of the main body 10. The internal hook 21 is attached to the carriage member 32 by a press or interference fit and extends along the length of the main body 10.

The carriage member 32 is attached to the washer, the washer is attached to the linear bushing 36, and the linear bushing 36 is attached to the plunger 31 to create a subassembly within the cavity 11 of the main body 10. The linear bushing 36 is designed to slow and control the speed of the push mechanism 30 and may be between 10 mm and 16 mm. These attachments can be made by any known to a person of ordinary skill in the art and can include press or interference fits or assembly by screws. The thumb rest 35 may be made of polycarbonate or other suitable biocompatible plastics such as polyoxymethylene (DELRIN), polyethylene, polycarbonate, and polyethylene terephthalate. Further, the thumb rest 35 preferably has an ergonomic shape having a concave surface to conform to the shape of a user's thumb. The overall length of the capsular tension ring insertion device 1 is preferably between 105 and 160 mm.

The spring 33 is preferably a spiral spring 33 and surrounds the internal hook 21 and is placed in the cavity 11 and rests between the shoulder member 34 of the push mechanism 30 and an internal surface 15 of the main body 10. The spiral spring 33 is configured to be in two states: an uncompressed state, which is the spring 33's resting state and a compressed state. In the uncompressed state, the plunger 31 and thumb rest 35 are fully extended outside of the cavity 11 of the main body 10. Additionally, the internal hook 21 is withdrawn into the cavity 11 of the main body 10, guided by the carriage member 32. This first position 33a is the default state of the capsular tension ring insertion device 1 when it is resting on a level surface.

When the user wishes to use the capsular tension ring insertion device 1 and prepare the capsular tension ring for insertion into the patient's intraocular space, the user picks up the capsular tension ring insertion device 1 and first inserts the external hook 22 into an eyelet 41 of the capsular tension ring. The user then pushes on the thumb rest 35 with their thumb to activate the push mechanism 30 and move the spring 33 from the uncompressed state to its compressed state. During this motion, the carriage member 32 guides the internal hook 21 along the length of the main body 10 allowing its tip to enter the tube 12 and extend beyond the distal end of the tube 12. At this second position 33b, the plunger 31 is within the cavity 11 of the main body 10, the thumb rest 35 is flush against an outer surface 16 of the main body 10, and the internal hook 21 is fully extended.

At this point, the user can then insert the internal hook 21 into another eyelet 42 of the capsular tension ring at its other end and slowly begin to release the push mechanism 30. As the user puts less pressure on the push mechanism 30, the spring 33 slowly returns to its uncompressed state and the internal hook 21 begins to withdraw back into the tube 12 and the main body 10 with the capsular tension ring attached guided by the carriage member 32 until all of the components are back to the first position 33a. However, in one variant of the capsular tension ring insertion device 1, the end of the main body 10 has a locking mechanism, which interacts with the push mechanism 30 to keep the push mechanism 30, the internal hook 21, and the spring 33 all maintained in the second position 33b. Thereby this feature does not require the user to maintain constant pressure on the push mechanism 30 to maintain it in the second position 33b.

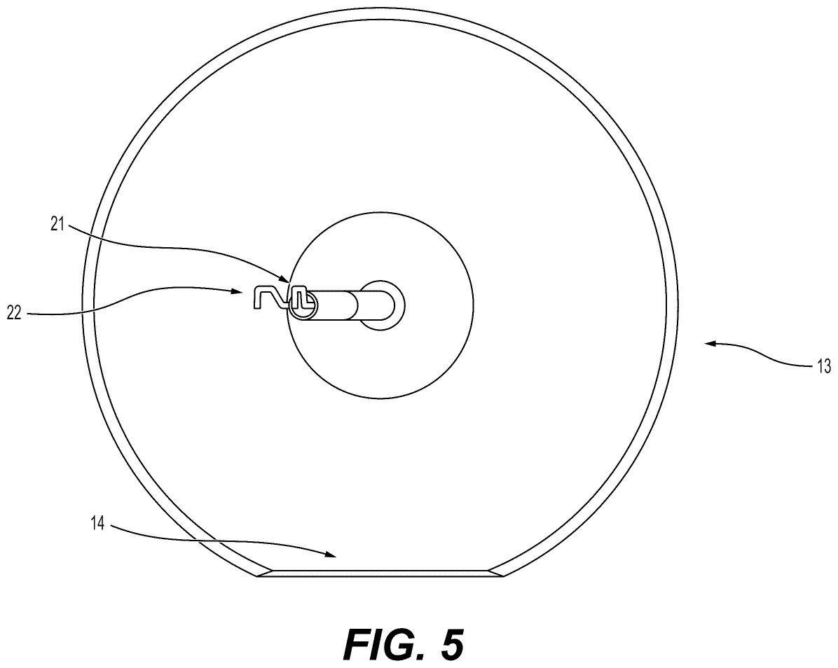

In another aspect of the invention, the main body 10 of the capsular tension ring insertion device 1 has a support structure 13 which can be used as a finger grip. This support structure 13 allows the user to put leverage with two fingers, preferably index and middle fingers, for greater control as the user controls the push mechanism 30. Thus, this capsular tension ring insertion device 1 can reliably be used with one hand without putting stress on the Zinn's membrane when the capsular tension ring insertion device 1 is inserted into a patient's intraocular space. Preferably, the support structure 13 has a length of 18 mm to 22 mm and may be in the shape of a circle, rectangle α, star, or another regular polygonal shape. Turning now to FIG. 5, this support structure 13 also has a flat portion 14 which prevents the capsular tension ring insertion device 1 from rolling around when it is placed on a level surface. Preferably, the flat portion 14 also acts a guide during the procedure. In one variant, the flat portion 14 is on a side of the capsular tension ring insertion device 1 closest to which the tip of the tube 12 curves. In another variant, the flat portion 14 is in a plane parallel to the plane in which the tip of the tube 12 curves. Thus, when using the capsular tension ring insertion device 1, the user can reliably know where the curvature of the tube 12 is and properly orient the capsular tension ring insertion device 1 during the procedure.

The present invention also relates to the method of inserting a capsular tension ring (seen in FIG. 8) into an intraocular space of a patient's eye using the insertion capsular tension ring insertion device 1. The steps of which are generally described in FIG. 9. First, a user, such as a licensed physician, presses on the push mechanism 30 of the capsular tension ring insertion device 1 to extend the internal hook 21 of the capsular tension ring insertion device 1 from the first position 33a to the second position 33b beyond the end of a tube 12 of the capsular tension ring insertion device 1. Next in S1, the user inserts the internal hook 21 into a first eyelet 41 of the capsular tension ring. Then in S2, the user pulls back on the push mechanism 30 to withdraw the internal hook 21 and the capsular tension ring into the tube 12 until the internal hook 21 and the first eyelet 41 are at a position approximately at a midpoint along a length of the tube 12 (i.e., the third position). The user may use forceps to help in the preparation process. The user then inserts external hook 22 of the capsular tension ring insertion device 1 into a second eyelet 42 of the capsular tension ring.

Following in S3, the user pulls on the push mechanism 30 to withdraw the internal hook 21 and the capsular tension ring into the tube 12 until the internal hook 21 is in the first position 33a. Thereafter, in S4, the user places a tip of the external hook 22 into the intraocular space of the eye. Preferably, the external hook 22 is placed at the center of the capsular bag located in the center of the patient's eye. Finally, in S5 and S6, the user presses on the push mechanism 30 to extend the internal hook 21 and the first eyelet 41 to the second position 33b and tilts the capsular tension ring insertion device 1 to release the capsular tension ring from the capsular tension ring insertion device 1. During this motion, the user gradually moves the capsular tension ring insertion device 1 toward the outer perimeter of the capsular bag spirally. This motion is known as a spiral method because the end of the capsular tension ring on the external hook 22 as it moves outward forms a spiral shape while the internal hook 21 releases the capsular tension ring from the main body 10 and the tube 12.

The capsular tension ring insertion device 1 may be a single use device (i.e., disposable after use) or reusable after appropriate sterilization. If reused, the capsular tension ring insertion device 1 preferably has a tip at the end of the plunger 31 where the thumb rest 35 is placed, which may be hooked up with a tube and flushed out after the thumb rest 35 is removed. As a result, the plunger 31 may be hollow to facilitate cleaning. Many forms of sterilization exist within the art and the sterilization methods can include steam, ethylene oxide, or gamma ray sterilization, among others.

While a capsular tension ring insertion device and corresponding method of inserting a capsular tension ring into an eye have been described in terms of what may be considered to be specific aspects, the present invention is not limited to the disclosed aspects. Additional modifications and improvements to the capsular tension ring insertion device and corresponding method may be apparent to those skilled in the art. Moreover, the many features and advantages of the disclosure are apparent from the detailed specification, and thus, it is intended by the appended claims to cover all such features and advantages of the present invention which fall within the spirit and scope of the disclosure. Further, it is not desired to limit the disclosure to the exact construction and operation illustrated and described, and accordingly, all suitable modifications and equivalents may be resorted to, falling within the scope of the disclosure. The present disclosure should therefore be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed:

1. A capsular tension ring insertion device for inserting a capsular tension ring having a pair of spaced apart eyelets into an intraocular space of an eye, the capsular tension ring insertion device comprising:
a main body having a first end and a second end;
a tube extending from the first end of the main body, the tube including a bore configured to receive the capsular tension ring, and the tube having a distal end defining an opening configured to allow the capsular tension ring to pass therethrough;
an external hook fixedly attached to the distal end of the tube, the external hook extending from an outer surface of the tube and beyond the distal end of the tube, a distal end of the external hook defining a first eyelet grip;
a push mechanism including a carriage member movably disposed within the main body and an actuator connected to the carriage member; and
an internal hook having a proximal end connected to the carriage member and a distal end defining a second eyelet grip, the internal hook being moveable with respect to the tube;
wherein the distal end of the internal hook is disposed within the tube when the push mechanism is in a first position; and
wherein the distal end of the internal hook extends beyond the distal end of the tube when the push mechanism is in a second position.

2. The capsular tension ring insertion device of claim 1, wherein the distal end of the external hook extends a first distance from the distal end of the tube and the distal end of the internal hook extends a second distance from the distal end of the tube when the push mechanism is in the second position, the second distance being greater than the first distance.

3. The capsular tension ring insertion device of any one of the preceding claims, further comprising a spring positioned within a cavity of the main body.

4. The capsular tension ring insertion device of claim 3, wherein the spring is configured to be in an uncompressed state when the push mechanism is in the first position.

5. The capsular tension ring insertion device of claim 3, wherein the spring is configured to be in a compressed state when the push mechanism is in the second position.

6. The capsular tension ring insertion device of any one of the preceding claims, wherein the tube has a curvature.

7. The capsular tension ring insertion device of claim 6, wherein the curvature has a radius of 5 mm to 30 mm.

8. The capsular tension ring insertion device of any one of the preceding claims, wherein an angle formed between the external hook and the internal hook is 5° to 45° when the push mechanism is in the second position.

9. The capsular tension ring insertion device of any one of the preceding claims, wherein at least one of the internal hook and the external hook comprise the shape of a fish-hook, a C-shape, or an L-shape.

10. The capsular tension ring insertion device of any one of the preceding claims, wherein a tip of at least one of the internal hook and the external hook comprises a ball shape, a tear-drop shape, or a loop.

11. The capsular tension ring insertion device of any one of the preceding claims, wherein the main body comprises a hollow cylinder and made of metal.

12. The capsular tension ring insertion device of any one of the preceding claims, wherein the main body is made of a metal.

13. The capsular tension ring insertion device of any one of the preceding claims, wherein the external hook and the internal hook are made of a metal.

14. The capsular tension ring insertion device of any one of the preceding claims, wherein the main body further comprises a support structure.

15. The capsular tension ring insertion device of claim 14, wherein the support structure includes a flat portion configured to prevent the device from rotating when the device is placed on a level surface.

16. The capsular tension ring insertion device of claim 14, wherein the support structure includes a flat portion operable to allow the user to guide the tube into the intraocular space of the eye.

17. The capsular tension ring insertion device of any one of the preceding claims, wherein the actuator includes a plunger having a distal end connected to the carriage member, and a proximal end connected to a push member.

18. A method for inserting a capsular tension ring into an intraocular space of an eye, the method comprising:
providing the capsular tension ring insertion device of claim 1;
extending the internal hook from a first position to a second position beyond an end of a tube of the device;
inserting the internal hook into a first eyelet of the capsular tension ring;
withdrawing the internal hook and the capsular tension ring into the tube until the internal hook and the first eyelet are at a third position;
inserting an external hook of the device into a second eyelet of the capsular tension ring;
withdrawing the internal hook and the capsular tension ring into the tube until the internal hook is in the first position;
inserting a tip of the external hook into the intraocular space of the eye;
actuating the push mechanism to extend the internal hook and the first eyelet of the capsular tension ring to the second position and spirally unwinding the capsular tension ring within the intraocular space; and
tilting the capsular tension ring insertion device to release the capsular tension ring.

19. The method of claim 18, wherein the third position is approximately at a midpoint along a length of the tube.

20. The method of any one of claims 18 and 19, where the tip of the external hook is inserted into the center of the eye.
